(19) United States
(12) Patent Application Publication
(10) Pub. No.: US 2011/0104084 A1
(43) Pub. Date: May 5, 2011

(54) NOVEL COMPOSITIONS BASED ON ETHERS OF ALKYL POLYOSIDES AND ON ALKYL GLYCERYL ETHERS, USE THEREOF AS AN EMULSIFIER AND COSMETIC COMPOSITIONS CONTAINING SAME

(75) Inventors: Sébastien Kerverdo, Vincennes (FR); Hervé Rolland, Castres (FR); Jerôme Guilbot, Castres (FR); Alain Milius, Nice (FR)

(73) Assignee: Societe D'Exploitation De Products Pour Les Industries Chimiques Seppic

(21) Appl. No.: 12/995,584

(22) PCT Filed: Jun. 15, 2009
(86) PCT No.: PCT/FR09/51127

§ 371 Date: Dec. 1, 2010

(30) Foreign Application Priority Data
Jun. 20, 2008 (FR) 0854086

(51) Int. Cl. A61K 8/60; A61Q 17/04

(52) U.S. Cl. 424/59

(57) ABSTRACT

Composition (C) comprising, per 100% of its weight: —from 10 wt % to 95 wt %, more particularly from 70 wt % to 95 wt % of one or more alcohol ethers of glycerol of formula (II): in which R1 represents a linear or branched, saturated or unsaturated aliphatic radical, optionally substituted with one or more hydroxyl groups, comprising from 16 to 18 carbon atoms, and—from 90 wt % to 5 wt %, more particularly from 30 wt % to 5 wt % of one or more compounds of formula (I): in which p represents a decimal number between 1 and 5, G represents the residue of a reducing sugar, R₁ represents an aliphatic radical as defined previously; its use for improving the stability of the dihydroxyacetone contained in a cosmetic composition, in particular a cosmetic composition intended for artificial browning and/or tanning of the skin.

The invention relates to the provision of novel nonionic emulsifying compositions that may be used in the preparation of cosmetic and/or pharmaceutical formulations especially for topical use, which are in the form of oil-in-water emulsions, which are stable on storage for a prolonged time under a temperature constraint.

Sugar-based emulsifiers have been developed for more than twenty years and their commercial success requires no further demonstration. These commercial compositions formed from a mixture of alkyl polyglycosides and of fatty alcohols are available on the market either in solid form, whether it is a case of flakes or pearls, or in a form that is liquid at room temperature if branched or unsaturated fatty alcohols are used to give such mixtures. Such emulsifying compositions are described in the international patent applications published under the numbers WO 92/06778, WO 95/13863, WO 96/37285 or WO 00/56438, or in the French patent application published under the number FR 2 830 464.

They are used for preparing cosmetic and/or pharmaceutical formulations that are in the form of water-in-oil and oil-in-water emulsions.

The preparation of cosmetic and/or pharmaceutical emulsions, which are in oil-in-water form, from commercial compositions comprising alkyl polyglycosides and fatty alcohols is well known to those skilled in the art, but their commercialization is hampered by their instability over time under storage conditions with a temperature constraint.

One solution to this problem lies in the use of an emulsifying composition comprising alkyl polyglycosides, fatty alcohols and alkyl sulfates and/or alkyl ether sulfates, as described and claimed in European patent application EP 0 771 559 A1. However, the use of such anionic constituents in emulsifying compositions intended for the preparation of cosmetic and/or pharmaceutical emulsions is preferentially not selected since the presence of anionic surfactants may induce cutaneous intolerance reactions when the emulsion is applied to the skin for a prolonged time. Moreover, the presence of anionic surfactants in the emulsifying composition does not make it possible to produce stable cosmetic and/or pharmaceutical emulsions with the combination of active agents usually used in the cosmetic and pharmaceutical industries, more particularly positively charged active agents.

The European patent application published under the number EP 0 550 280 A1 describes glyceroglycolipid surfactants of formula:

A₁-O—CH₂—CH(OR₁)—CH₂—OR (a)

in which A1 is a saccharide residue, R and R1, which may be identical or different, represent a hydrogen atom or a linear or branched, saturated or unsaturated hydrocarbon-based radical containing from 1 to 24 carbon atoms, it being understood that at least two radicals do not represent a hydrogen atom, and also their use for preparing cleansing or detergent compositions for fabrics or hard surfaces, and also personal care compositions for cleansing the human body, for instance solid soaps, face or body cleansing compositions, hair or body shampoos and toothpastes. That patent application more particularly discloses glyceroglycolipid surfactants such as those of formula (a) in which A1 represents a galactose, glucose, mannose, lactose, galactotrioside or cellotrioside residue, R and R1 represent different alkyl chains. On the other hand, that patent application does not disclose the use of these surfactants as emulsifiers for preparing oil-in-water emulsions that are stable on storage for a prolonged time under a temperature constraint.

The French patent application published under the number FR 2 765 876 discloses hydrophilic glycosides of formula:

R₁—O—CH₂—CH(OR₂)—CH₂—O[G]ₚ

in which R1 represents an optionally hydroxyl-functionalized alkyl, alkenyl or acyl radical containing from 6 to 22 carbon atoms, R2 represents a hydrogen or R1, G represents a saccharide radical with 5 to 12 carbon atoms and p represents numbers ranging from 1 to 10, and also a process for preparing them that consists in etherifying the glycoses with partial ethers of glycerol in the presence of alkaline catalysts and at temperatures ranging from 100° C. to 180° C. In Example 4 thereof, the preparation process includes a step of reaction between glucose and 2-ethylhexyl glyceryl ether under acid catalysis, and also a purification step consisting in distilling the 2-ethylhexyl glyceryl ether.

The inventors have thus sought to develop novel nonionic emulsifying compositions for preparing cosmetic and/or pharmaceutical oil-in-water emulsions for topical application, which are stable on storage for a prolonged time under a temperature constraint. This is why, according to a first aspect, a subject of the invention is a composition (C) comprising, per 100% of its mass:
from 10% by mass to 95% by mass and more particularly from 70% by mass to 95% by mass of one or more glyceryl alkyl ethers of formula (II):

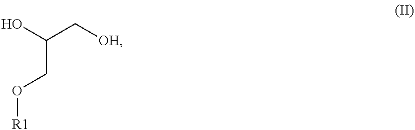

in which R1 represents a linear or branched, saturated or unsaturated aliphatic radical optionally substituted with one or more hydroxyl groups, containing from 16 to 18 carbon atoms, and
from 90% by mass to 5% by mass and more particularly from 30% by mass to 5% by mass of one or more compounds of formula (I):

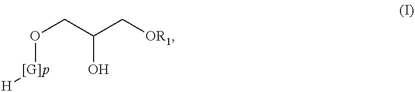

in which p represents a decimal number between 1 and 5, G represents a reducing sugar residue, R₁ represents an aliphatic radical as defined previously.

The term “linear or branched, saturated or unsaturated aliphatic radical optionally substituted with one or more hydroxyl groups containing from 16 to 18 carbon atoms” especially denotes for R₁ in formulae (I) and (II) as defined above:
linear aliphatic radicals, for example n-hexadecyl and n-octadecyl radicals;
aliphatic radicals derived from isoalkanols of formula (A):

(CH₃)(CH₃)CH—(CH₂)ₘ—CH₂—OH (A)
in which m represents an integer between 12 and 14, for example isohexadecyl or isooctadecyl radicals;
branched aliphatic radicals derived from Guerbet alcohols of formula (B):

CH(CₛH₂ₛ₊₁)(CₜH₂ₜ₊₁)—CH₂—OH (B)
in which t is an integer between 8 and 12, s is an integer between 2 and 8 and the sum s+t is greater than or equal to 14, for example 2-ethyltetradecyl, 2-ethylhexadecyl, 2-butyltetradecyl, 2-hexyldodecyl, 2-octyldecyl or 2-hexyldecyl radicals;
unsaturated linear aliphatic radicals such as hexadecenyl, octadecenyl, octadecadienyl, octadeca-trienyl and octadecatetraenyl radicals, for example oleyl, isooleyl or linoleyl unsaturated radicals;
saturated or unsaturated, linear or branched aliphatic radicals containing from 16 to 18 carbon atoms substituted with one or two hydroxyl groups, such as hydroxyhexadecyl, hydroxyoctadecyl or dihydroxyoctadecyl radicals, for example 3-hydroxyhexadecyl, 4-hydroxyhexadecyl acid, 11-hydroxyhexadecyl acid, 16-hydroxyhexadecyl acid, 12-hydroxystearyl acid or 8,9-dihydroxystearyl radicals.

In the definition of the compounds of formula (I) as defined previously, p is a decimal number that represents the average degree of polymerization of the residue G. When p is an integer, (G)p is the polymer residue of rank p of the residue G. When p is a decimal number, formula (I) represents a mixture of compounds: a₁ R₁—O—CH₂—CH(OR₂)—CH₂—O-G-H+a₃ R₁—O—CH₂—CH(OR₂)—CH₂—O-(G)₂-H+a₃ R₁—O—CH₂—CH(OR₂)—CH₂—O-(G)₃-H+ . . . +a_{q} R₁—O—CH₂—CH(OR₂)—CH₂—O-(G)_{q}-H with q representing an integer between 1 and 10 and in the molar proportions a₁, a₂, a₃ . . . a_{q} such that:

$${\sum }_{q = 10}^{q - 1} {a}_{q} = 1 ; {a}_{1} > 0$$

According to another particular aspect of the present invention, in the definition of the compounds of formula (I), p is between 1.05 and 5 and more particularly between 1.05 and 2.

The term “reducing sugar residue” denotes for G in the definition of the compound of formula (I) a residue of saccharide derivatives that do not contain in their structures a glycoside bond established between an anomeric carbon and the oxygen of an acetal group as defined in the reference publication: “Biochemistry”, Daniel Voet/Judith G. Voet, p. 250, John Wiley & Sons, 1990. The oligomeric structure (G)ₚ may be in any isomeric form, whether it is a case of optical isomerism, geometrical isomerism or positional isomerism; it may also represent a mixture of isomers.

In formula (I) as defined above, the group R₁—O—CH₂—CH(OH)—CH₂—O— is connected to G via the anomeric carbon of the saccharide residue, so as to form an acetal function.

In the context of the present invention, the reducing sugar residue G is more particularly chosen from the group formed by glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose.

According to a more particular aspect of the present invention, in formulae (I) and (II) as defined above, the radical R₁ is chosen from hexadecyl, 2-hexyldecyl, octadecyl, oleyl, 12-hydroxystearyl, 2-hexyldodecyl and 2-octyldecyl radicals.

According to another more particular aspect of the present invention, in formula (I) as defined above, G represents a reducing sugar residue chosen from glucose, xylose and arabinose.

A subject of the invention is also a process for preparing a composition (C) as defined above, comprising a step a) of reacting a reducing sugar G with a stoichiometric excess of alkyl glyceryl ether of formula (II) as defined previously.

In the process for preparing composition (C), step a) is generally performed in a reactor, by controlling the stoichiometric ratio between the two reactants, and by mechanical stirring under predetermined temperature and partial vacuum conditions, for example at a temperature of between 70° C. and 130° C. and under a partial vacuum of between 300 mbar (3×10⁴ Pa) and 20 mbar (2×10³ Pa).

The term “acidic catalytic system” denotes strong acids such as sulfuric acid, hydrochloric acid, phosphoric acid, nitric acid, hypophosphorous acid, methanesulfonic acid, para-toluenesulfonic acid, trifluoromethanesulfonic acid or acidic ion-exchange resins.

A subject of the invention is also a variant of the process as defined previously, comprising a step a1) of reacting a reducing sugar G with an alcohol of formula (III)

R₃—OH (III),

in which R₃ represents a linear aliphatic radical containing from 1 to 4 carbon atoms, and more particularly with butanol, in the presence of an acidic catalytic system, to form the acetal of formula (IV):

R₃O-(G)ₚ (IV),

in which p represents a decimal number between 1 and 5; followed by a step b) of transacetalization of the acetal of formula (IV) obtained in step a1), by adding an excess of alkyl glyceryl ether of formula (II) as defined previously with distillation under vacuum of the alcohol of formula (III) formed in situ.

Step a1) of the variant of the process as defined above is more particularly performed at a temperature of between 90° C. and 105° C., under partial vacuum, and is often accompanied by the concomitant removal of the water formed during the reaction. As catalytic system used in this step a1), it is more particularly the system as defined for performing the preparation process from which said variant is derived.

The process and its variant may, if necessary or if desired, be completed by neutralization, filtration and decolorization operations.

Composition (C) as defined previously may also be prepared by mixing an alkyl glyceryl ether of formula (II) or a mixture of alkyl glyceryl ethers of formula (II) with a compound of formula (I), or a mixture of compounds of formula (I), prepared separately beforehand according to the process or its variant as described previously, which include, however, a final additional step of removing the excess alkyl glyceryl ether(s) by performing an operation known to those skilled in the art, for instance distillation, distillation under a partial vacuum using a thin-film evaporator or a direct-path thin-film evaporator, molecular distillation or solvent extraction.

A subject of the invention is also a composition (C′) comprising, per 100% of its mass:
from 90% by mass to 5% by mass of a composition (C1) comprising, per 100% of its mass, from 10% to 90% by mass of at least one compound of formula (I_{A}) corresponding to formula (I) in which R₁ represents a radical containing 18 carbon atoms, and from 95% to 5% by mass of at least one compound of formula (I_{B}) corresponding to formula (I), in which R₁ represents a radical containing 16 carbon atoms; and
from 10% by mass to 95% by mass of a mixture of alkyl glyceryl ethers of formula (IIA) corresponding to formula (II) in which R₁ represents a radical containing 18 carbon atoms, and alkyl glyceryl ethers of formula (IIB) corresponding to formula (II) for which R₁ represents a radical containing 16 carbon atoms.

Composition (C′) as defined above may be prepared by mixing composition (C₁) as defined above with at least one alkyl glyceryl ether of formula (II_{A}) as defined above and at least one alkyl glyceryl ether of formula (II_{B}) as defined above.

Composition (C₁) may be prepared via various routes.

A first preparation route for composition (C₁) consists in mixing, in the desired mass proportions, the compound of formula (I_{A}) as defined above or a mixture of compounds of formula (I_{A}), with the compound of formula (I_{B}) as defined above, or a mixture of compounds of formula (I_{B}).

A second preparation route for composition (C₁) consists in performing a process comprising a step a) of reacting a reducing sugar G with a mixture (M₁) comprising, per 100% of its total mass, from 10% to 90% by mass of at least one alkyl glyceryl ether of formula (II_{A}), corresponding to formula (II) for which the radical R₁ represents an aliphatic radical containing 18 carbon atoms, and from 90% to 10% by mass of at least one alkyl glyceryl ether of formula (II_{B}), corresponding to formula (II) for which the radical R₁ represents an aliphatic radical containing 16 carbon atoms, in the presence of an acidic catalytic system. In this second route for preparing composition (C₁), step a) is generally performed in a reactor, by controlling the stoichiometric ratio between the two reagents, and with mechanical stirring under predetermined temperature and partial vacuum conditions, for example at a temperature of between 70° C. and 130° C. and under a partial vacuum of between 300 mbar (3×10⁴ Pa) and 20 mbar (2×10³ Pa).

The term “acidic catalytic system” denotes strong acids such as sulfuric acid, hydrochloric acid, phosphoric acid, nitric acid, hypophosphorous acid, methanesulfonic acid, para-toluenesulfonic acid, trifluoromethanesulfonic acid or acidic ion-exchange resins.

A third route for preparing composition (C₁) consists of a variant of the second preparation route as described previously, comprising a step a1) of reacting a reducing sugar G with an alcohol of formula (III) as defined previously, in the presence of an acidic catalytic system, to form the acetal of formula (IV), followed by a step b) of transacetalization of the acetal of formula (IV) obtained in step a1), by adding a mixture (M1) as defined previously with distillation under vacuum of the alcohol of formula (III) formed in situ.

Step a1) of this third preparation route as described above is more particularly performed at a temperature of between 90° C. and 105° C., under partial vacuum, and it is often accompanied by the concomitant removal of the water formed during the reaction.

As catalytic system used in this step a1), there is more particularly the system as defined for performing the second preparation route from which said variant is derived.

In the second preparation route and its variant, the excess mixture (M1) is removed by performing an operation known to those skilled in the art, for instance distillation, distillation under partial vacuum using a thin-film evaporator or a direct-path thin-film evaporator, molecular distillation or solvent extraction. The second preparation route and its variant may, if necessary or if desired, be completed by neutralization, filtration and decolorization operations.

Composition (C′) as defined above may also be prepared by reacting an excess of mixture (M₁) as defined previously with the reducing sugar G according to the second preparation route or its variant, as defined previously, without removing the alkyl glyceryl ethers of formulae (II_{A}) and (II_{B}) included in the unreacted mixture (M₁).

A subject of the invention is also the use of a composition (C) or of a composition (C′) as defined previously, as emulsifier.

A subject of the invention is also a composition for topical use that is in the form of an oil-in-water emulsion comprising, per 100% of its mass:
from 0.1% by mass to 15% by mass of a composition (C) or of a composition (C′) as defined previously,
from 5% by mass to 50% by mass of a fatty phase formed by one or more oils and/or one or more waxes, and
from 94.9% by mass to 35% by mass of a cosmetically acceptable medium.

The expression “for topical use” used in the definition of the composition in the form of an oil-in-water emulsion as described above means that said composition is used by application to the skin, the hair, the scalp or mucous membranes, whether it is a matter of direct application in the case of a cosmetic, dermocosmetic, dermopharmaceutical or pharmaceutical composition or indirect application, for example in the case of a body hygiene product in the form of a textile or paper wipe or sanitary products intended to come into contact with the skin or mucous membranes.

Among the oils that may be used in the fatty phase of the composition in the form of an oil-in-water emulsion that is the subject of the present invention, mention may be made of mineral oils such as liquid paraffin, liquid petroleum jelly, isoparaffins or white mineral oils; oils of animal origin, such as squalene or squalane; plant oils, such as phytosqualane, sweet almond oil, coconut oil, castor oil, jojoba oil, olive oil, rapeseed oil, groundnut oil, sunflower oil, wheatgerm oil, corn germ oil, soyabean oil, cotton oil, alfalfa oil, poppy oil, pumpkin oil, evening primrose oil, millet oil, barley oil, rye oil, safflower oil, candlenut oil, passionflower oil, hazelnut oil, palm oil, Shea butter, apricot kernel oil, beauty-leaf oil, sysymbrium oil, avocado oil, calendula oil, oils obtained from flowers or legumes; ethoxylated plant oils; synthetic oils, for instance fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides, such as glyceryl triheptanoate, alkyl benzoates, hydrogenated oils, poly-α-olefins, polyolefins such as polyisobutene, synthetic isoalkanes such as isohexadecane, identified in Chemical Abstracts by the number RN=93685-80-4 and which is a mixture of C₁₂, C₁₆ and C₂₀ isoparaffins containing at least 97% of C₁₆ isoparaffins, among which the main constituent is 2,2,4,4,6,8,8-heptamethylnonane (RN=4390-04-9), isododecane, such as hydrogenated polydecene or hydrogenated polyisobutene, sold in France by the company Ets B. Rossow et Cie under the name Parleam-Polysynlane™, mentioned in Michel and Irene Ash; Thesaurus of Chemical Products, Chemical Publishing Co, Inc. 1986 Volume 1, page 211 (ISBN 0 7131 3603 0), perfluoro oils; and silicone oils such as dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alkyl groups.

Among the waxes that may be used in the fatty phase of the composition in the form of an oil-in-water emulsion that is the subject of the present invention, mention may be made of beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax, silicone waxes; plant waxes; fatty alcohols and fatty acids that are solid at room temperature; glycerides that are solid at room temperature.

As another fatty substance that may be combined with the fatty phase of the composition in the form of an oil-in-water emulsion that is the subject of the present invention, mention may be made of saturated or unsaturated, linear or branched fatty alcohols, and saturated or unsaturated, linear or branched fatty acids.

The term “cosmetically acceptable medium” used in the definition of the composition in the form of an oil-in-water emulsion as described above and which is the subject of the present invention means, according to the directive of the Council of the European Economic Community No. 76/768/EEC of Jul. 27, 1976 modified by directive No. 93/35/EEC of Jun. 14, 1993, any substance or preparation intended to come into contact with the various parts of the human body (epidermis, bodily and head hair system, nails, lips and genital organs) or with the teeth and the visible oral mucosae, exclusively and principally, to cleanse them, perfume them, modify their appearance and/or correct body odors and/or protect them or keep them in good condition. A cosmetically acceptable medium for these compositions that are the subject of the invention may conventionally contain water, one or more cosmetically acceptable organic solvents, or a mixture of water and one or more organic solvents.

The cosmetically acceptable solvents may be chosen more particularly from polyhydric alcohols, for instance glycerol, diglycerol, glycerol oligomers, ethylene glycol, propylene glycol, hexylene glycol, diethylene glycol, xylitol, erythritol, sorbitol, or water-soluble alcohols such as ethanol, isopropanol or butanol.

According to another particular aspect, the composition for topical use according to the invention that is in the form of an oil-in-water emulsion also comprises, per 100% of its mass, from 0.1% by mass to 5% by mass of a thickening and/or gelling polymer.

Among the thickening and/or gelling polymers that may be combined with the composition for topical use that is in the form of an oil-in-water emulsion as defined previously and that is the subject of the present invention, mention may be made of polymers of polyelectrolyte type, for instance copolymers of acrylic acid and of 2-methyl[(1-oxo-2-propenyl)amino]-1-propanesulfonic acid (AMPS), copolymers of acrylamide and of 2-methyl[(1-oxo-2-propenyl)amino]-1-propane-sulfonic acid, copolymers of 2-methyl[(1-oxo-2-propenyl)amino]-1-propanesulfonic acid and of (2-hydroxyethyl)acrylate, 2-methyl[(1-oxo-2-propenyl)-amino]-1-propanesulfonic acid homopolymer, acrylic acid homopolymer, copolymers of acryloylethyltrimethyl-ammonium chloride and of acrylamide, copolymers of AMPS and vinylpyrrolidone, copolymers of acrylic acid and of alkyl acrylates whose carbon chain comprises between ten and thirty carbon atoms, copolymers of AMPS and of alkyl acrylates whose carbon chain comprises between ten and thirty carbon atoms. Such polymers are sold, respectively under the names Simulgel™ EG, Sepigel™ 305, Simulgel™ NS, Simulgel™ 800, Simulgel™ A, Simulgel™ EPG, Simulgel™ INS, Simulgel™ FL, Simulgel SMS 88, Sepigel™ 501, Sepigel™ 502, Sepiplus™ 250, Sepiplus™ 265, Sepiplus™ 400, Sepiplus S, Sepinov™ EMT 10, Carbopol™, Ultrez™ 10, Aculyn™, Pemulen™ TR1, Pemulen™ TR2, Luvigel™ EM, Salcare™ SC91, Salcare™ SC92, Salcare™ SC95, Salcare™ SC96, Flocare™ ET100, Flocare™ ET58, Hispagel™, Novemer™ EC1, Aristoflex™ AVC, Aristoflex™ HBM, Rapithix™ A60, Rapithix™ A100, Cosmedia SP and Stabileze™ 06; hydrocolloids of plant or biosynthetic origin, for example xanthan gum, karaya gum, carrageenates, alginates, galactomannans; silicates; cellulose and derivatives thereof; starch and hydrophilic derivatives thereof; polyurethanes.

According to another more particular aspect, the composition for topical use according to the invention that is in the form of a water-in-oil emulsion also comprises, per 100% of its mass, from 0.5% by mass to 10% by mass of dihydroxyacetone.

Dihydroxyacetone is a product commonly used in cosmetics as an agent for artificially tanning and/or browning the skin; when applied to the skin it affords a tanning or browning effect whose appearance is more or less similar to that which may result from prolonged exposure to sunlight or under an ultraviolet radiation lamp. The composition for topical use that is in the form of an oil-in-water emulsion comprising, per 100% of its mass, from 0.5% to 10% by mass of dihydroxyacetone and that is the subject of the present invention may also be combined with other skin tanning and/or browning agents, among which mention may be made of monocarbonyl or polycarbonyl compounds, for instance isatin, alloxan, ninhydrin, glyceraldehyde, mesotartaric aldehyde, glutaraldehyde and erythrulose.

A subject of the invention is also a cosmetic treatment process for artificially tanning and/or browning the skin, characterized in that it consists in applying to the skin an effective amount of the composition as defined above.

A subject of the invention is also the use of a composition (C) or of a composition (C′) as defined previously, for improving the stability of the dihydroxyacetone contained in a cosmetic composition for artificially tanning and/or browning the skin.

The compositions for topical use that are in the form of an oil-in-water emulsion as defined previously and that are the subject of the present invention may be prepared in the following manner:
the aqueous phase is heated to a temperature of between 60° C. and 85° C.;
in parallel, the fatty phase comprising the emulsifying system that is the subject of the present invention and the oils and/or waxes is heated to a temperature of between 60° C. and 85° C.

The two phases are then mixed together and emulsified using an emulsifier of rotor-stator type (for instance a Silverson or Ultra-Turrax laboratory emulsifier). After emulsification for a few minutes, the emulsion is cooled with moderate stirring at a speed of between 50 rpm and 200 rpm. The thickening and/or gelling polymer or the mixtures of thickening and/or gelling polymers are introduced into the aqueous phase, or into the oily phase or directly into the emulsion according to the manufacturers' recommendations. If the components of each phase or of all the phases are liquid at room temperature, the preparation of said phase or of the emulsion may be performed without heating.

The compositions for topical use that are in the form of an oil-in-water emulsion as defined previously and that are the subjects of the present invention may be in the form of creams, milks, cream gels, fluid lotions or vaporizable fluid lotions.

In general, these compositions for topical use that are in the form of oil-in-water emulsions as defined previously also comprise excipients and/or active principles usually used in the field of formulations for topical use, in particular cosmetic, dermocosmetic, pharmaceutical or dermopharmaceutical formulations, such as thickening and/or gelling surfactants, stabilizers, film-forming compounds, hydrotropic agents, plasticizers, emulsifiers and coemulsifiers, opacifiers, nacreous agents, overfatting agents, sequestrants, chelating agents, antioxidants, fragrances, preserving agents, conditioning agents, bleaching agents for decolorizing bodily hair and the skin, active agents intended to give the skin or the hair a treating action, sunscreens, mineral fillers or pigments, particles that afford a visual effect or that are intended for encapsulating active agents, exfoliant particles, texturing agents, optical brighteners and insect repellants.

As examples of thickening and/or gelling surfactants that may be present in the compositions for topical use that are in the form of oil-in-water emulsions that are the subject of the present invention, mention may be made of optionally alkoxylated fatty esters of alkylpolyglycosides, and more particularly ethoxylated methylpolyglucoside esters such as PEG 120 methyl glucose trioleate and PEG 120 methyl glucose dioleate sold, respectively, under the names Glucamate™ LT and Glumate™ DOE120; alkoxylated fatty esters such as PEG 150 pentaerythrityl tetrastearate sold under the name Crothix™ DS53, PEG 55 propylene glycol oleate sold under the name Antil™ 141; fatty-chain polyalkylene glycol carbamates such as PPG 14 laureth isophoryl dicarbamate sold under the name Elfacos™ T211, PPG 14 palmeth 60 hexyl dicarbamate sold under the name Elfacos™ GT2125.

As examples of emulsifiers that may be present in the compositions for topical use that are in the form of oil-in-water emulsions that are the subject of the present invention, mention may be made of optionally alkoxylated alkylpolyglycoside fatty esters, and most particularly of ethoxylated methylpolyglucoside esters such as PEG 120 methyl glucose trioleate and PEG 120 methyl glucose dioleate sold, respectively, under the names Glucamate™ LT and Glumate™ DOE120; alkoxylated fatty esters such as PEG 150 pentaerythrityl tetrastearate sold under the name Crothix™ DS53, PEG 55 propylene glycol oleate sold under the name Antil™ 141; fatty-chain polyalkylene glycol carbamates such as PPG laureth isophoryl dicarbamate sold under the name Elfacos™ T211, PPG 14 palmeth 60 hexyl dicarbamate sold under the name Elfacos™ GT2125; fatty acids, ethoxylated fatty acids, fatty acid esters of sorbitol, fatty acid esters of mannitol, ethoxylated fatty acid esters, polysorbates, polyglyceryl esters, ethoxylated fatty alcohols, sucrose esters, alkylpolyglycosides, fatty alcohol sulfates and phosphates or mixtures of alkylpolyglycosides and of fatty alcohols described in the French patent applications published under the numbers 2 668 080, 2 734 496, 2 756 195, 2 762 317, 2 784 680, 2 784 904, 2 791 565, 2 790 977, 2 807 435, 2 804 432, 2 830 774, 2 830 445, combinations of emulsifying surfactants chosen from alkylpoly-glycosides, combinations of alkylpolyglycosides and of fatty alcohols, polyglyceryl or polyglycol or polyol esters such as the polyglycol or polyglycerol polyhydroxystearates used in the French patent applications published under the numbers 2 852 257, 2 858 554, 2 820 316 and 2 852 258.

As examples of opacifiers and/or nacreous agents that may be present in the compositions for topical use that are in the form of oil-in-water emulsions that are the subject of the present invention, mention may be made of sodium or magnesium palmitates or stearates or hydroxystearates, ethylene or polyethylene glycol monostearates or distearates, fatty alcohols, styrene homopolymers and copolymers such as the styrene acrylate copolymer sold under the name Montopol™ OP1 by the company SEPPIC.

As examples of active principles that may be present in the compositions for topical use that are in the form of oil-in-water emulsions that are the subject of the present invention, mention may be made of: compounds with a lightening or depigmenting action, for instance arbutin, kojic acid, hydroquinone, ellagic acid, vitamin C or vitamin C derivatives, for instance magnesium ascorbyl phosphate, polyphenol extracts, grape extracts, pine extracts, wine extracts, olive extracts, marc extracts, apple juice extracts; amino acid derivatives, for instance the undecelenoyl-phenylalanine sold under the name Sepiwhite MSH, peptides; total protein hydrolyzates; partial protein hydrolyzates; polyols (for instance glycerol or butylene glycol); urea; pyrrolidonecarboxylic acid or derivatives of this acid; glycyrrhetinic acid; α-bisabolol; sugars or sugar derivatives; polysaccharides or derivatives thereof, hydroxy acids, for instance lactic acid; vitamines or vitamin derivatives, for instance retinol, vitamin E and derivatives thereof; mineral salts; enzymes; coenzymes, for instance coenzyme Q10; hormones or “hormone-like” compounds, for instance Phyto-Age™; soyabean extracts, for instance Raffermine™; wheat extracts, for instance Tensine™ or Gliadine™; plant extracts such as tannin-rich extracts, isoflavone-rich extracts or terpene-rich extracts; extracts of freshwater algae or marine algae; extracts of marine plants; essential waxes; bacterial extracts; lipids in general and more particularly lipids such as ceramides or phospholipids; active agents with antimicrobial activity or a purifying action on greasy skin; active agents with an energizing or stimulating property, for instance Sepitonic™ M3 or Physiogenyl™; panthenol and derivatives thereof such as Sepicap™ MP; anti-aging active agents, for instance Sepivinol™, Sepivital™, Extramel™ C and Manoliva™; moisturizing active agents such as Aquaxyl™; “anti-photoaging” active agents; active agents with an immediate tensioning or smoothing action on the skin, for instance Sesaflash™; active agents for protecting the integrity of the dermoepidermal junction, for instance Phyto-age™; active agents for increasing the synthesis of extracellular matrix components, for instance Sepitonic™ M3 and Aquaxyl™; active agents with slimming, firming or draining activity, for instance caffeine, caffeine derivatives, theophylline, cyclic adenosine monophosphate (cAMP), Adipoless™, green tea, sage, Ginkgo biloba, ivy, common horse chestnut, bamboo, ruscus, butcher's-broom, Centella asiatica, meadowsweet, fucus, rosemary, willow, parsnip extracts, potentilla extracts; active agents that create a “heating” sensation on the skin, such as cutaneous capillary circulation activators, for instance nicotinates; active agents that create a “freshness” sensation on the skin, for instance menthol and derivatives thereof; active agents with action on stem cells; active agents with action on the epidermis, the dermis, the hypodermis and cutaneous appendages (bodily hair, nails, sebaceous glands, pores, etc.); active agents with an action on the cutaneous flora.

As examples of sunscreens that may be present in the composition for topical use that is in the form of an oil-in-water emulsion that is the subject of the present invention, mention may be made of any of those included in the cosmetic directive 76/768/EEC modified by annex VII.

The examples that follow illustrate the invention without, however, limiting it.

### A)—Preparation of Compositions According to the Invention

### EXAMPLE 1

### Preparation of a Composition (X) Formed from n-hexadecyl glyceryl ether and n-hexadecyl glyceryl ether polyglucosides

300.0 g of n-hexadecyl glyceryl ether (or chimyl alcohol) are placed in a jacketed glass reactor, in which circulates a heat-exchange fluid, and equipped with an efficient stirrer, at a temperature of 80° C. to enable total melting of the n-hexadecyl glyceryl ether. 31.5 g of anhydrous glucose are then gradually added to the reaction medium to allow its homogeneous dispersion. The homogeneous mixture is maintained at a temperature of 80° C. for 30 minutes, and 0.3 g of 98% sulfuric acid and 0.25 g of 50% hypophosphorous acid are then introduced into the homogeneous dispersion prepared beforehand. The reaction medium is placed under a partial vacuum of 90 mbar to 45 mbar, and maintained at a temperature of 100° C.-105° C. for a period of 5 hours with removal of the water formed by means of distillation apparatus. The reaction medium is then cooled to 85° C.-90° C. and neutralized by adding 0.3 g of 40% sodium hydroxide to bring the pH of a 5% solution of this mixture to a value of about 6.5.

### EXAMPLE 2

### Preparation of a Composition Y Formed from n-octadecyl glyceryl ether and n-octadecyl glyceryl ether polyglucosides

324.0 g of n-octadecyl glyceryl ether (or batyl alcohol) are placed in a jacketed glass reactor, in which circulates a heat-exchange fluid, and which is equipped with an efficient stirrer, at a temperature of 80° C. to enable total melting of the n-octadecyl glyceryl ether. 31.5 g of anhydrous glucose are then gradually added to the reaction medium to allow its homogeneous dispersion. The homogeneous mixture is maintained at a temperature of 80° C. for 30 minutes, and 0.3 g of 98% sulfuric acid and 0.25 g of 50% hypophosphorous acid are then introduced into the homogeneous dispersion prepared beforehand. The reaction medium is placed under a partial vacuum of 90 mbar to 45 mbar, and maintained at a temperature of 100° C.-105° C. for a period of 5 hours with removal of the water formed by means of distillation apparatus. The reaction medium is then cooled to 85° C.-90° C. and neutralized by adding 0.3 g of 30% sodium hydroxide to bring the pH of a 5% solution of this mixture to a value of about 6.5.

### EXAMPLE 3

### Preparation of a Composition Z Formed from n-hexadecyl glyceryl ether, n-octadecyl glyceryl ether, n-hexadecyl glyceryl ether polyglucosides and n-octadecyl glyceryl ether polyglucosides

149.7 g of n-hexadecyl glyceryl ether and 161.4 g of n-octadecyl glyceryl ether are placed in a jacketed glass reactor, in which circulates a heat-exchange fluid and which is equipped with an efficient stirrer, at a temperature of 80° C. to enable total melting of the alkyl glyceryl ethers. 31.5 g of anhydrous glucose are then gradually added to the reaction medium to allow its homogeneous dispersion. The homogeneous mixture is maintained at a temperature of 80° C. for 30 minutes, and 0.3 g of 98% sulfuric acid and 0.25 g of 50% hypophosphorous acid are then introduced into the homogeneous dispersion prepared beforehand. The reaction medium is placed under a partial vacuum of 90 mbar to 45 mbar, and maintained at a temperature of 100° C.-105° C. for a period of 5 hours with removal of the water formed by means of distillation apparatus. The reaction medium is then cooled to 85° C.-90° C. and neutralized by adding 0.3 g of 30% sodium hydroxide to bring the pH of a 5% solution of this mixture to a value of about 6.5. The analytical characteristics of the three compositions (X), (Y) and (Z) prepared previously are collated in Table 1 below:

TABLE 1
Analytical characteristics of compositions
(X), (Y) and (Z).

| | Composition | Composition | Composition |
|---|---|---|---|
| | (X) | (Y) | (Z) |
| Appearance at 20° C. | Beige- | Beige- | Beige- |
| (Visual determination) | colored | colored | colored |
| | solid | solid | solid |
| Acid number (IA) | 0.2 | 0.2 | 0.2 |
| (mg KOH/g) | | | |
| (Analysis according to | | | |
| standard NFT 60204) | | | |
| Hydroxyl number (IOH) | 396 | 371 | 385 |
| (mg KOH/g) | | | |
| (Analysis according to | | | |
| standard USP XXI NF XVI | | | |
| 01/01/1995) | | | |
| pH at 5% in water | 7.1 | 6.1 | 6.5 |
| Water (mass %) | 0.2 | 0.1 | 0.2 |
| (Analysis according to | | | |
| standard NFT 73201) | | | |
| Melting point (° C.) (Mettler | 57 | 66 | 61 |
| block) | | | |
| Chimyl alcohol (mass %) | 78.7 | / | 40.2 |
| Determination by gas | | | |
| chromatography | | | |
| Batyl alcohol (mass %) | / | 78.6 | 40.4 |
| Gas chromatography | | | |

### B)—Demonstration of the Improvement in Stability of Cosmetic Compositions in the Form of Oil-in-Water Emulsions, Over a Prolonged Time and Under a Temperature Constraint, in the Presence of Compositions According to the Invention

Several series of oil-in-water emulsions are prepared, the compositions of which are given in Tables 2 to 5 below, using in the emulsifying system compositions (X), (Y) and (Z) according to the invention in comparison with a prior art emulsifier formed from cetearyl polyglucosides and cetearyl alcohol, sold under the name Montanov™ 68 by the company SEPPIC. Emulsions E1 to E23 contained in Examples 4 to 7 are prepared according to the following common procedure:

The fatty phase is introduced into a reactor and brought to a temperature of 80° C. The emulsifying system is then introduced into the fatty phase at a temperature of 80° C. and the mixture obtained is homogenized for 30 minutes by means of a stirrer equipped with a paddle of anchor type, at a speed of 80 rpm. The water is then added at a temperature of 80° C., and the resulting mixture is sheared by means of an emulsifying blender of rotor-stator type, sold by the company Silverson, for a perid of 4 minutes at a speed of 4000 rpm. The preserving agent is then introduced into the emulsified mixture thus obtained, which is then cooled to room temperature and stirred for a further 10 minutes using a stirrer equipped with a paddle of anchor type, at a speed of 80 rpm. When the emulsion comprises a thickening and/or gelling polymer in its composition, it is introduced into the mixture formed from the fatty phase and the emulsifying system at a temperature of 80° C., before the step of adding water. The emulsions prepared are then stored in an insulated climatic chamber regulated to a temperature of 20° C., for 7 days. After this period of 7 days, the appearance of the prepared emulsion is observed and the viscosity of the emulsion is measured. It will be considered for the purposes of the experimental demonstration that the viscosity of the water-in-oil emulsion measured after 7 days at 20° C. is characteristic of a system at thermodynamic equilibrium, thus constituting a point of reference for the destabilization that may be generated by storage for a longer time at a higher temperature. The emulsions are then stored in an insulated climatic chamber regulated at a temperature of 45° C. for a period of 3 months. After a period of one month, the emulsions are removed from the climatic chamber to measure the viscosity, and are then returned to the same climatic chamber to continue the storage at a temperature of 45° C.

### EXAMPLE 4

### Demonstration of the Improvement in Stability of Oil-in-Water Emulsions Comprising an Apolar Oil Phase in the Absence of Thickening and/or Gelling Polymers

TABLE 2
Composition and characterization of emulsions
E1 to E3.

| | Emulsion E1 | Emulsion E2 | Emulsion E3 |
|---|---|---|---|
| Emulsifying system | | | |
| Montanov ™ 68 | 3% | 0% | 3% |
| Composition (Y) | 0% | 3% | 0% |
| Fatty phase: Primol ™ 352 | 10% | 10% | 20% |
| Preserving agent: Sepicide ™ HB | 0.5% | 0.5% | 0.5% |
| Water | qs 100% | qs 100% | qs 100% |
| Viscosity at 7 days at 20° C. | 11 200 mPa · s | 4000 mPa · s | 20 000 mPa · s |
| [Brookfield LVT M6 V6 | | | |
| viscometer (spindle 6, | | | |
| speed: 6 rpm)] | | | |
| Viscosity at 1 month at 45° C. | 3700 mPa · s | 6700 mPa · s | Dephasing |
| [Brookfield LVT M5 V6 | | | |
| viscometer (spindle 5, | | | |
| speed: 6 rpm)] | | | |
| Appearance after 3 months at | Homogeneous | Homogeneous | Dephasing |
| 45° C. | | | |

Composition and characterization
of emulsions E4 to E6.

| | Emulsion E4 | Emulsion E5 | Emulsion E6 |
|---|---|---|---|
| Emulsifying system | | | |
| Montanov ™ 68 | 0% | 5% | 0% |
| Composition (Y) | 3% | 0% | 5% |
| Fatty phase: Primol ™ 352 | 20% | 20% | 20% |
| Preserving agent: Sepicide ™ HB | 0.5% | 0.5% | 0.5% |
| Water | qs 100% | qs 100% | qs 100% |
| Viscosity at 7 days at 20° C. | 5500 mPa · s | 21 000 mPa · s | 24 000 mPa · s |
| (Brookfield LVT M6 V6) | | | |
| Viscosity at 1 month at 45° C. | 6400 mPa · s | 18 500 mPa · s | 16 500 mPa · s |
| (Brookfield LVT M6 V6) | | | |
| Appearance after 3 months at | Homogeneous | Homogeneous | Homogeneous |
| 45° C. | | | |

These results reveal that the oil-in-water emulsions comprising an apolar phase, prepared with composition (Y) according to the invention, are stable after storage for a period of 3 months at a temperature of 45° C. Furthermore, the use of composition (Y) according to the invention makes it possible to stabilize the viscosity value of the oil-in-water emulsion prepared with an apolar phase after storage at 45° C. for a period of 3 months: thus, the viscosity of emulsion E1, comprising the cetearyl polyglucosides/cetearyl alcohol mixture as emulsifier, decreases by 66.9% after one month of storage at 45° C., whereas the viscosity of emulsion E2 increases.

### EXAMPLE 5

### Demonstration of the Improvement in Stability of Oil-in-Water Emulsions Comprising a Polar Oil Phase in the Absence of Thickening and/or Gelling Polymers

TABLE 3
Composition and characterization of emulsions
E7 to E12.

| | Emulsion E7 | Emulsion E8 | Emulsion E9 |
|---|---|---|---|
| Emulsifying system | | | |
| Montanov ™ 68 | 3% | 0% | 0% |
| Composition (Y) | 0% | 0% | 3% |
| Composition (Z) | 0% | 0% | 0% |
| Composition (X) | 0% | 3% | 0% |
| Fatty phase: C8-C10 triglyceride | 10% | 10% | 10% |
| Preserving agent: Sepicide ™ HB | 0.5% | 0.5% | 0.5% |
| Water | qs 100% | qs 100% | qs 100% |
| Viscosity at 7 days at 20° C. | 13 200 mPa · s | 3700 mPa · s | 2410 mPa · s |
| (Brookfield LVT M5 V6) | | | |
| Viscosity at 1 month at 45° C. | Dephasing | 525 mPa · s | 4100 mPa · s |
| (Brookfield LVT M5 V6) | | | |
| Appearance after 3 months at | Dephasing | Homogeneous | Homogeneous |
| 45° C. | from 1 month | emulsion | emulsion |
| | at 45° C. | | |
| | Emulsion E10 | Emulsion E11 | Emulsion E12 |
| Emulsifying system | | | |
| Montanov ™ 68 | 3% | 0% | 0% |
| Composition (Y) | 0% | 3% | 0% |
| Composition (Z) | 0% | 0% | 3% |
| Composition (X) | 0% | 0% | 0% |
| Fatty phase: C8-C10 triglyceride | 50% | 50% | 50% |
| Preserving agent: Sepicide ™ HB | 0.5% | 0.5% | 0.5% |
| Water | qs 100% | qs 100% | qs 100% |
| Viscosity at 7 days at 20° C. | 15 600 mPa · s | 11 200 mPa · s | 11 100 mPa · s |
| (Brookfield LVT M5 V6) | | | |
| Viscosity at 1 month at 45° C. | Dephasing | 6900 mPa · s | 6360 mPa · s |
| (Brookfield LVT M5 V6) | | | |
| Appearance after 3 months at | Dephasing | Homogeneous | Homogeneous |
| 45° C. | from 7 days | emulsion | emulsion |
| | at 45° C. | | |

These results reveal that the oil-in-water emulsions comprising a polar phase, prepared with compositions (X), (Y) and (Z) according to the invention, are stable and homogeneous after storage for a period of 3 months at a temperature of 45° C., whereas the comparative emulsions comprising the cetearyl polyglucosides/cetearyl alcohol mixture as emulsifier dephase after one month of storage at a temperature of 45° C.

### EXAMPLE 6

### Demonstration of the Improvement in Stability of Oil-in-Water Emulsions Comprising an Apolar Oil Phase in the Presence of Thickening and/or Gelling Polymers

TABLE 4
Composition and characterization of emulsions
E13 to E15.

| | Emulsion E13 | Emulsion E14 | Emulsion E15 |
|---|---|---|---|
| Emulsifying system | | | |
| Montanov ™ 68 | 1% | 0% | 5% |
| Composition (Y) | 0% | 1% | 0% |
| Composition (Z) | 0% | 0% | 0% |
| Composition (X) | 0% | 0% | 0% |
| Fatty phase | | | |
| Primol ™ 352 | 20% | 20% | 20% |
| Simulgel ™ 600 | 0.2% | 0.2% | 0.2% |
| Preserving agent | | | |
| Sepicide ™ HB | 0.5% | 0.5% | 0.5% |
| Water | qs 100% | qs 100% | qs 100% |
| Viscosity at | 8100 mPa · s | 2010 mPa · s | 34 000 mPa · s |
| 7 days at 20° C. | | | |
| (Brookfield | | | |
| LVT M5 V6) | | | |
| Viscosity at | Dephasing | 2300 mPa · s | 21 000 mPa · s |
| 1 month at 45° C. | | | |
| (Brookfield | | | |
| LVT M5 V6) | | | |
| Appearance after | Dephasing | Homogeneous | Homogeneous |
| 3 months at 45° C. | | emulsion | emulsion |

Composition and characterization
of emulsions E16 and E17

| | Emulsion E16 | Emulsion E17 |
|---|---|---|
| Emulsifying system | | |
| Montanov ™ 68 | 0% | 0% |
| Composition (Y) | 0% | 5% |
| Composition (Z) | 0% | 0% |
| Composition (X) | 5% | 0% |
| Fatty phase | | |
| Primol ™ 352 | 20% | 20% |
| Simulgel ™ 600 | 0.2% | 0.2% |
| Preserving agent | | |
| Sepicide ™ HB | 0.5% | 0.5% |
| Water | qs 100% | qs 100% |
| Viscosity at 7 days at 20° C. | 40 800 mPa · s | 18 640 mPa · s |
| (Brookfield LVT M6 V6) | | |
| Viscosity at 1 month at 45° C. | 18 200 mPa · s | 22 000 mPa · s |
| (Brookfield LVT M6 V6) | | |
| Appearance after 3 months at | Homogeneous | Homogeneous |
| 45° C. | emulsion | emulsion |

These results reveal that the oil-in-water emulsions comprising an apolar phase, in the presence of a thickening polymer, prepared with composition (Y) according to the invention are stable and homogeneous after storage for a period of 3 months at a temperature of 45° C. When compared with emulsion E14, emulsion E13 comprising the same mass content of 1% of the emulsifier formed by the cetearyl polyglucosides/cetearyl alcohol mixture dephases before one month of storage at a temperature of 45° C. Furthermore, the viscosity of emulsion E15, comprising the cetearyl polyglucosides/cetearyl alcohol mixture as emulsifier, decreases by 38% after one month of storage at 45° C., whereas over the same period and under the same storage conditions, the viscosity of emulsion E17 comprising composition (Y) according to the invention increases.

### EXAMPLE 7

### Demonstration of the Improvement in Stability of Oil-in-Water Emulsions Comprising a Polar Oil Phase in the Presence of Thickening and/or Gelling Polymers

TABLE 5
Composition and characterization of emulsions
E18 to E23.

| | Emulsion E18 | Emulsion E19 | Emulsion E20 |
|---|---|---|---|
| Emulsifying system | | | |
| Montanov ™ 68 | 1% | 0% | 0% |
| Composition (Y) | 0% | 1% | 0% |
| Composition (Z) | 0% | 0% | 1% |
| Composition (X) | 0% | 0% | 0% |
| Fatty phase | | | |
| C8-C10 triglyceride | 20% | 20% | 20% |
| Simulgel ™ 600 | 0.2% | 0.2% | 0.2% |
| Preserving agent: Sepicide ™ HB | 0.5% | 0.5% | 0.5% |
| Water | qs 100% | qs 100% | qs 100% |
| Viscosity at 7 days at 20° C. | 4100 mPa · s | 2600 mPa · s | 2380 mPa · s |
| (Brookfield LVT M5 V6) | | | |
| Viscosity at 1 month at 45° C. | Dephasing | 1800 mPa · s | 1800 mPa · s |
| (Brookfield LVT M5 V6) | | | |
| Appearance after 3 months at | Dephasing | Homogeneous | Homogeneous |
| 45° C. | from 1 month | emulsion | emulsion |
| | at 45° C. | | |
| | Emulsion E21 | Emulsion E22 | Emulsion E23 |
| Emulsifying system | | | |
| Montanov ™ 68 | 5% | 0% | 0% |
| Composition (Y) | 0% | 0% | 5% |
| Composition (Z) | 0% | 0% | 0% |
| Composition (X) | 0% | 5% | 0% |
| Fatty phase | | | |
| C8-C10 triglyceride | 20% | 20% | 20% |
| Simulgel ™ 600 | 0.2% | 0.2% | 0.2% |
| Preserving agent: Sepicide ™ HB | 0.5% | 0.5% | 0.5% |
| Water | qs 100% | qs 100% | qs 100% |
| Viscosity at 7 days at 20° C. | 38 500 mPa · s | 46 000 mPa · s | 30 500 mPa · s |
| (Brookfield LVT M6 V6) | | | |
| Viscosity at 1 month at 45° C. | 25 200 mPa · s | 39 000 mPa · s | 35 000 mPa · s |
| (Brookfield LVT M6 V6) | | | |
| Appearance after 3 months at | Homogeneous | Homogeneous | Homogeneous |
| 45° C. | emulsion | emulsion | emulsion |

The results of series 4 reveal that the oil-in-water emulsions comprising a polar phase, in the presence of a thickening polymer, prepared with a mass content of 1% of emulsifier formed by compositions (Y) and (Z) according to the invention are stable and homogeneous after storage for a period of 3 months at a temperature of 45° C. (emulsions E19 and E20, respectively), whereas the comparative oil-in-water emulsion (emulsion E18) prepared with the same mass content of emulsifier formed by the cetearyl polyglucosides/cetearyl alcohol mixture dephases before one month of storage at a temperature of 45° C. Furthermore, the use of a mass content of 5% of composition (Y) according to the invention as emulsifier for preparing an oil-in-water emulsion (emulsion E23) makes it possible to stabilize and to increase the viscosity of the emulsion after one month of storage at 45° C., whereas the viscosity of the comparative emulsion (emulsion E21), comprising the same mass content of 5% of the emulsifier formed by the cetearyl polyglucosides/cetearyl alcohol mixture, decreases by 34.5% and dephases after the same period of one month of storage at a temperature of 45° C.

### EXAMPLE 8

### Demonstration of the Improvement in Color Stability of Oil-in-Water Self-Tanning Emulsions

Dihydroxyacetone has the drawback of degrading over time, leading to problems of storage over time that are generally reflected by undesired yellowing of the compositions containing it.

### a) Principle of the Method for Measuring the Color of a Cosmetic Composition

The change in coloration of compositions comprising dihydroxyacetone is estimated by the comparison over time of a numerical characteristic ΔE, calculated from the measurement of three constituent parameters of a color:
the parameter L*, ranging between 0 and 100, which represents the lightness of the shade; the higher the value, the lighter the shade
the parameter a*, which ranges from −60 to +60, and expresses the entire range from green (a*=−60) to red (a*=+60)
the parameter b*, which ranges from −60 to +60 and expresses the entire range from blue (b*=−60) to yellow (b*=+60).

The parameters L*, a* and b*, characterizing an oil-in-water emulsion comprising dihydroxyacetone, are measured for different storage times (t1, t2) of said oil-in-water emulsion at a given temperature, and the value ΔE is then calculated as follows:

ΔE=√((ΔL*)²+(Δa*)²+(Δb*)²),

with:

ΔL*=L* at t2−L* at t1

Δa*=a* at t2−a* at t1

Δb*=a* at t2−b* at t1

### b) Experimental Protocol

Several oil-in-water emulsions are prepared, the compositions of which are indicated in the table below, using in the emulsifying system compositions (Y) and (Z) according to the invention in comparison with a prior art emulsifier formed from cetearyl polyglucosides and cetearyl alcohol, sold under the name Montanov™ 68 by the company SEPPIC.

Emulsions E24 to E26 are prepared according to the following common procedure:
the components of the fatty phase are melted separately at a temperature of 80° C., and then introduced into a reactor and homogenized at a temperature of 80° C. for 15 minutes using a stirrer equipped with a paddle of “anchor” type at a stirring speed of 80 rpm;
the aqueous phase is prepared in parallel in a beaker by mixing, at a temperature of 80° C., the water, the glycerol and the propylene glycol;
the thickening polymer is added to the fatty phase prepared previously, and the resulting mixture is then homogenized at a temperature of 80° C. for 15 minutes;
the aqueous phase is added to the mixture comprising the fatty phase and the thickening polymer;
the resulting mixture is then sheared using an emulsifying machine of “rotor-stator” type sold by the company Silverson, for a duration of 4 minutes at a speed of 4000 rpm, at a temperature of 80° C.;
the emulsion obtained is then stirred for 30 minutes using a stirrer equipped with a paddle of “anchor” type at a stirring speed of 100 rpm, and then allowed to cool;
when the temperature reaches 40° C., the solution of dihydroxyacetone at 50% in water is added along with the preserving agents;
when the temperature reaches 20° C., the emulsion obtained is stirred for 15 minutes using a stirrer equipped with a paddle of “anchor” type at a stirring speed of 100 rpm.

### c) Experimental Results

The compositions of each emulsion E24 to E26 are indicated in table 6 below, as is the characterization of their appearance and of their change in coloration (ΔE) after storage for 3 months at 45° C. The parameters L*, a* and b*, which constitute the color of each emulsion, are measured with a Minolta CR200 chromameter sold by the company Minolta, so as to measure the corresponding value ΔE.

TABLE 6
Composition and characterization of emulsions
E24 to E26.

| | Emulsion E24 | Emulsion E25 | Emulsion E26 |
|---|---|---|---|
| Emulsifying system | | | |
| Montanov ™ 68 | 2% | 0% | 0% |
| Composition (Y) | 0% | 2% | 0% |
| Composition (Z) | 0% | 0% | 2% |
| Fatty phase | | | |
| C12-C15 | 10% | 10% | 10% |
| alkylbenzoate | | | |
| DC 200/350⁽⁵⁾ | 5% | 5% | 5% |
| Thickening polymer: | 0.35% | 0.35% | 0.35% |
| Simulgel ™ INS 100 | | | |
| Preserving agents | | | |
| Sepicide ™ HB | 1.0% | 1.0% | 1.0% |
| Tocopherol | 0.05% | 0.05% | 0.05% |
| Aqueous phase | | | |
| Glycerol | 3.0% | 3.0% | 3.0% |
| Propylene glycol | 2.0% | 2.0% | 2.0% |
| Water | qs 100% | qs 100% | qs 100% |
| Self-tanning agent | | | |
| Dihydroxyacetone | 10.0% | 10.0% | 10.0% |
| as a 50% solution | | | |
| in water | | | |
| Appearance of the | Homogeneous | Homogeneous | Homogeneous |
| emulsions after | emulsion | emulsion | emulsion |
| 3 months at 45° C. | | | |
| ΔE after 3 months | 6.8 | 2.6 | 3.5 |
| at 45° C. | | | |

The results of Example 8 show that the oil-in-water emulsions comprising dihydroxyacetone, prepared with compositions (Y) and (Z) according to the invention, give rise to a reduced change in coloration after storage for a period of 3 months at a temperature of 45° C. when compared with the comparative emulsion comprising as emulsifier the cetearyl polyglucosides/cetearyl alcohol mixture. Thus, the value of the characteristic ΔE after storage for 3 months at a temperature of 45° C. is 6.8 for the comparative emulsion E24, whereas the same characteristic ΔE is, respectively, 2.6 and 3.5 for emulsions E25 and E26 according to the invention, i.e. a respective reduction of 61.8% and 48.5% in the increase in coloration.

### C)—Formulations

In the following formulations, the percentages are expressed as mass percentages per 100% of the mass of the formulation.

### EXAMPLE 9

### Care Cream

| | |
|---|---|
| Cyclomethicone: | 10% |
| Simulgel ™ EG: | 0.8% |
| Composition Y: | 2% |
| Stearyl alcohol: | 1% |
| Stearic alcohol: | 0.5% |
| Preserving agent: | 0.65% |
| Lysine: | 0.025% |
| EDTA (disodium salt): | 0.05% |
| Xanthan gum: | 0.2% |
| Glycerol: | 3% |
| Water: | qs 100% |

### EXAMPLE 10

### Antisun Milk

FORMULA

| | | |
|---|---|---|
| A | Composition (Y): | 3.0% |
| | Sesame oil: | 5.0% |
| | Parsol ™ MCX: | 5.0% |
| | λ carrageenan: | 0.10% |
| B | Water: | qs 100% |
| C | Simulgel ™ NS: | 0.80% |
| D | Fragrance: | qs |
| | Preserving agent: | qs |

### Procedure

Emulsify B in A at 75° C., then add C at about 60° C., then D at about 30° C., and adjust the pH if necessary.

### EXAMPLE 11

### Moisturizing and Matting Foundation

FORMULA

| | | |
|---|---|---|
| A | Water: | 20.0% |
| | Butylene glycol: | 4.0% |
| | PEG-400: | 4.0% |
| | Pecosil ™ PS100: | 1.0% |
| | Sodium hydroxide: | qs pH = 9 |
| | Titanium dioxide: | 7.0% |
| | Talc: | 2.0% |
| | Yellow iron oxide: | 0.8% |
| | Red iron oxide: | 0.3% |
| | Black iron oxide: | 0.05% |
| B | Lanol ™ 99: | 8% |
| | Caprylic/capric triglyceride: | 8% |
| | Composition (Y): | 5.00: |
| C | Water: | qs 100% |
| | Micropearl ™ M305: | 2.0% |
| | Tetrasodium EDTA: | 0.05% |
| D | Cyclomethicone: | 4.0% |
| | Xanthan gum: | 0.2% |
| | Simulgel ™ EG: | 2.5% |
| E | Sepicide ™ HB: | 0.5% |
| | Sepicide CI: | 0.3% |
| | Fragrance: | 0.2% |

### Procedure

Prepare the mixtures B+D and A+C at 80° C., then mix together and emulsify the whole.

### EXAMPLE 12

### Body Milk

| | |
|---|---|
| Composition (Y): | 3.5% |
| Lanol ™ 37T: | 8.0% |
| Solagum ™ L: | 0.05% |
| Water: | qs 100% |
| Benzophenone-3: | 2.0% |
| Dimethicone 350 cPs: | 0.05% |
| Simulgel ™ NS: | 2.5% |
| Preserving agent: | 0.2% |
| Fragrance: | 0.4% |

### EXAMPLE 13

### Makeup-Removing Emulsion with Sweet Almond Oil

| | |
|---|---|
| Composition (Z): | 5% |
| Sweet almond oil: | 5% |
| Water: | qs 100% |
| Simulgel ™ INS 100: | 0.3% |
| Glycerol: | 5% |
| Preserving agent: | 0.2% |
| Fragrance: | 0.3% |

### EXAMPLE 14

### Moisturizing Cream for Greasy Skin

| | |
|---|---|
| Composition (Y): | 5% |
| Cetylstearyl octanoate: | 8% |
| Octyl palmitate: | 2% |
| Water: | qs 100% |
| Simulgel ™ NS: | 2.6% |
| Micropearl ™ M100: | 3.0% |
| Mucopolysaccharides: | 5% |
| Sepicide ™ HB: | 0.8% |
| Fragrance: | 0.3% |

### EXAMPLE 15

### Cream with AHA for Sensitive Skin

| Mixture of N-laurylamino acids: | 0.1% to 5% |
|---|---|
| Magnesium potassium aspartate: | 0.002% to 0.5% |
| Lanol ™ 99: | 2% |
| Composition (Y): | 5.0% |
| Water: | qs 100% |
| Simulgel ™ NS: | 1.50% |
| Gluconic acid: | 1.50% |
| Triethanolamine (TEA): | 0.9% |
| Sepicide ™ HB: | 0.3% |
| Sepicide ™ CI: | 0.2% |
| Fragrance: | 0.4% |

### EXAMPLE 16

### Makeup-Removing Milk

| | |
|---|---|
| Composition (X): | 3% |
| Primol ™ 352: | 8.0% |
| Sweet almond oil: | 2% |
| Water: | qs 100% |
| Simulgel ™ NS: | 0.8% |
| Preserving agent: | 0.2% |

### EXAMPLE 17

### Antisun Milk

| | |
|---|---|
| Composition (Y): | 3.5% |
| Lanol ™ 37T: | 10.0% |
| Parsol ™ MCX: | 5.0% |
| Eusolex ™ 4360: | 2.0% |
| Water: | qs 100% |
| Sepiplus ™ 400: | 1.8% |
| Preserving agent: | 0.2% |
| Fragrance: | 0.4% |

### EXAMPLE 18

### Cream with AHA

| | |
|---|---|
| Composition (Y): | 5.0% |
| Deepaline ™ PVB: | 1.05% |
| Lanol ™ 99: | 10.0% |
| Water: | qs 100% |
| Gluconic acid: | 1.5% |
| TEA (triethanolamine): | 0.9% |
| Simulgel ™ NS: | 1.5% |
| Fragrance: | 0.4% |
| Sepicide ™ HB: | 0.2% |
| Sepicide ™ CI: | 0.4% |

### EXAMPLE 19

### Self-Tanning Emulsion

| | |
|---|---|
| Lanol ™ 99: | 15% |
| Composition (Y): | 5.0% |
| Parsol ™ MCX: | 3.0% |
| Water: | qs 100% |
| Dihydroxyacetone: | 5.0% |
| Monosodium phosphate: | 0.2% |
| Simulgel ™ NS: | 2.5% |
| Fragrance: | 0.3% |
| Sepicide ™ HB: | 0.8% |
| Sodium hydroxide: | qs pH = 5 |

### EXAMPLE 20

### Care Cream

| | |
|---|---|
| Cyclomethicone: | 10% |
| Simulgel ™ EG: | 2.8% |
| Composition (Y): | 4.5% |
| Preserving agent: | 0.65% |
| Lysine: | 0.025% |
| EDTA (disodium salt): | 0.05% |
| Xanthan gum: | 0.2% |
| Glycerol: | 3% |
| Water: | qs 100% |

### EXAMPLE 21

### Care Cream

| | |
|---|---|
| Cyclomethicone: | 10% |
| Sepigel ™ 305: | 0.8% |
| Composition (Y): | 4.5% |
| Perfluoropolymethyl isopropyl ether: | 0.5% |
| Preserving agent: | 0.65% |
| Lysine: | 0.025% |
| EDTA (disodium salt): | 0.05% |
| Pemulen ™ TR1: | 0.2% |
| Glycerol: | 3% |
| Water: | qs 100% |

### EXAMPLE 22

### Body Milk

FORMULA

| | | |
|---|---|---|
| A | Composition (Z) | 3.0% |
| | Glyceryl triheptonate: | 10.0% |
| B | Water: | qs 100% |
| C | Simulgel ™ EG: | 1.0% |
| D | Fragrance: | qs |
| | Preserving agent: | qs |

### Procedure

Melt A at about 75° C. Emulsify B in A at 75° C. and then add C at about 60° C., followed by D.

### EXAMPLE 23

### Body Milk

| | |
|---|---|
| Composition (Y): | 3.5% |
| Lanol ™ 37T: | 8.0% |
| Solagum ™ L: | 0.05% |
| Water: | qs 100% |
| Benzophenone-3: | 2.0% |
| Dimethicone 350 cPs: | 0.05% |
| Simulgel ™ NS: | 2.8% |
| Preserving agent: | 0.2% |
| Fragrance: | 0.4% |

### EXAMPLE 24

### Cream with AHA

| | |
|---|---|
| Composition (Y): | 5.0% |
| Deepaline ™ PVB: | 1.05% |
| Lanol ™ 99: | 10.0% |
| Water: | qs 100% |
| Gluconic acid: | 1.5% |
| TEA (triethanolamine): | 0.9% |
| Simulgel ™ EG: | 1.5% |
| Fragrance: | 0.4% |
| Sepicide ™ HB: | 0.2% |
| Sepicide ™ CI: | 0.4% |

### EXAMPLE 25

### Makeup-Removing Milk

| | |
|---|---|
| Simulsol ™ 165: | 4% |
| Composition (Z): | 1% |
| Caprylate/caprate triglyceride | 15% |
| Pecosil ™ DCT: | 1% |
| Demineralized water: | qs |
| Capigel ™ 98: | 0.5% |
| Simulgel ™ INS 100: | 1% |
| Proteol ™ APL: | 2% |
| Sodium hydroxide: | qs pH = 7 |

### EXAMPLE 26

### Antisun Cream

| | |
|---|---|
| Simulsol ™ 165: | 3% |
| Composition (Y): | 2% |
| C12-C15 benzoate: | 8% |
| Pecosil ™ PS 100: | 2% |
| Dimethicone: | 2% |
| Cyclomethicone: | 5% |
| Octyl para-methoxycinnamate: | 6% |
| Benzophenone-3: | 4% |
| Titanium oxide: | 8% |
| Xanthan gum: | 0.2% |
| Butylene glycol: | 5% |
| Demineralized water: | qs 100% |
| Simulgel ™ NS: | 1.5% |
| Preserving agent, fragrance: | qs |

### EXAMPLE 27

### Vitamin-Containing Cream

| | |
|---|---|
| Simulsol ™ 165: | 5% |
| Composition (Y): | 1% |
| Caprylic/capric triglycerides: | 20% |
| Vitamin A palmitate: | 0.2% |
| Vitamin E acetate: | 1% |
| Micropearl ™ M 305: | 1.5% |
| Simulgel ™ 600: | 2% |
| Water: | qs 100% |
| Preserving agent, fragrance: | qs |

### EXAMPLE 28

### Antisun Self-Tanning Gel

| | |
|---|---|
| Composition (Y): | 3.0% |
| Glyceryl triheptanoate: | 10.0% |
| Deepaline ™ PVB: | 1.05% |
| Simulgel ™ EG: | 2.2% |
| Water: | qs 100% |
| Dihydroxyacetone: | 5% |
| Fragrance: | 0.1% |
| Sepicide ™ HB: | 0.3% |
| Sepicide ™ CI: | 0.1% |
| Parsol ™ MCX: | 4.0% |

### EXAMPLE 29

### Self-Tanning Cream Containing α-Hydroxy Acids

| | |
|---|---|
| Composition (Y): | 5.0% |
| Deepaline ™ PVB: | 1.05% |
| Lanol ™ 99: | 10.0% |
| Water: | qs 100% |
| Gluconic acid: | 1.5% |
| Dihydroxyacetone: | 3% |
| Triethanolamine: | 0.9% |
| Simulgel ™ EG: | 1.5% |
| Fragrance: | 0.4% |
| Sepicide ™ HB: | 0.2% |
| Sepicide ™ CI: | 0.4% |

### EXAMPLE 30

### Self-Tanning Cream Containing α-Hydroxy Acids for Sensitive Skin

| Mixture of N-lauroylamino acids: | 0.1% to 5% |
|---|---|
| Magnesium potassium aspartate: | 0.002% to 0.5% |
| Composition (X): | 5.0% |
| Lanol ™ 99: | 2.0% |
| Water: | qs 100% |
| Lactic acid: | 1.5% |
| Dihydroxyacetone: | 3.5% |
| Triethanolamine: | 0.9% |
| Simulgel ™ NS: | 1.5% |
| Fragrance: | 0.4% |
| Sepicide ™ HB: | 0.3% |
| Sepicide ™ CI: | 0.2% |

The definitions of the commercial products used in the examples are as follows:

Montanov™ 68 (cetearyl glucoside/cetearyl alcohol) is a self-emulsifying composition as described in WO 92/06778, sold by the company SEPPIC.
Capigel™ 98 is a liquid thickener based on an acrylate copolymer, sold by the company SEPPIC.
Lanol™ 99 is isononyl isononanoate sold by the company SEPPIC.
Micropearl™ M 100 is an ultrafine powder with a very soft feel and a matting action, sold by the company Matsumo.
Sepicide™ CI, imidazolidineurea, is a preserving agent sold by the company SEPPIC.
Pemulen™ TR1 is an acrylic polymer sold by Goodrich.
Simulsol™ 165 is self-emulsifying glyceryl stearate sold by the company SEPPIC.
Sepicide™ HB, which is a mixture of phenoxyethanol, methyl paraben, ethyl paraben, propyl paraben and butyl paraben, is a preserving agent sold by the company SEPPIC.
Parsol™ MCX is octyl para-methoxycinnamate; sold by the company Givaudan.
Lanol™ 37T is glyceryl triheptanoate, sold by the company SEPPIC.
Solagum™ L is a carrageenan sold by the company SEPPIC.
Eusolex™ 4360 is a sunscreen sold by the company Merck.
Deepaline™ PVB is an acylated wheat protein hydrolyzate sold by the company SEPPIC.
Proteol™ APL is a foaming surfactant sold by the company SEPPIC.
Micropearl™ M 305 is a silky water-dispersible powder based on crosslinked methyl methacrylate copolymer.
Simulgel™ EG: Self-inversible inverse latex of copolymer such as those described in the international publication WO 99/36445 (INCI name: sodium acrylate/sodium acryloyldimethyltaurate copolymer and isohexadecane and polysorbate 80), sold by the company SEPPIC.
Sepiplus™ 400: Self-inversible inverse latex of copolymers such as those described in the international publication WO 2005/040 230 (INCI name: polyacrylate-13 & polyisobutene & polysorbate 20), sold by the company SEPPIC.
Simulgel™ NS: Self-inversible inverse latex of thickening copolymers (INCI name: hydroxyethyl acrylate/sodium acryloyldimethyltaurate copolymer and squalane and polysorbate 60), sold by the company SEPPIC.
Simulgel™ INS 100: Self-inversible inverse latex of thickening copolymers (INCI name: hydroxyethyl acrylate/sodium acryloyldimethyltaurate copolymer and isohexadecane and polysorbate 60), sold by the company SEPPIC.
Simulgel™ 600: Self-inversible inverse latex of thickening copolymers (INCI name: acrylamide/sodium acryloyldimethyltaurate copolymer and isohexadecane and polysorbate 80), sold by the company SEPPIC.
Sepigel™ 305: Self-inversible inverse latex of thickening copolymers (INCI name: polyacrylamide and C13-C14 isoparaffin and laureth-7) sold by the company SEPPIC.
Primol™ 352 is a mineral oil sold by the company Exxon.
Pecosil™ DCT is sodium dimethicone PEG-7 acetyl methyl taurate sold by the company Phoenix.
Pecosil™ PS 100 is dimethicone PEG-7 sold by the company Phoenix.
DC 200/350 is a cyclomethicone sold by the company Dow Corning.

1. A composition (C) comprising, per 100% of its mass:
from 10% by mass to 95% by mass and more particularly from 70% by mass to 95% by mass of one or more alkyl glyceryl ethers of formula (II): 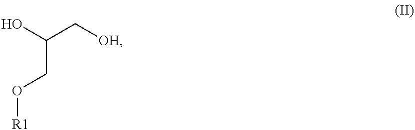
in which R1 represents a linear or branched, saturated or unsaturated aliphatic radical optionally substituted with one or more hydroxyl groups, comprising from 16 to 18 carbon atoms, and
from 90% by mass to 5% by mass and more particularly from 30% by mass to 5% by mass of one or more compounds of formula (I): 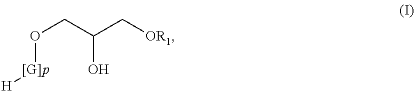
in which p represents a decimal number between 1 and 5, G represents a reducing sugar residue and R₁ represents an aliphatic radical as defined previously.

2. The composition (C) as defined in claim 1,
characterized in that, in formulae (I) and (II), R₁ represents a radical chosen from hexadecyl, 2-hexyldecyl, octadecyl, oleyl, 12-hydroxystearyl, 2-hexyldodecyl and 2-octyldecyl radicals.

3. The composition (C) as defined in claim 1, characterized in that, in formula (I), G represents a reducing sugar residue chosen from glucose, xylose and arabinose.

4. A composition C′ corresponding to composition (C) as defined in claim 1, comprising, per 100% of its mass:
from 90% by mass to 5% by mass of a composition (C1) comprising from 10% to 90% by mass of at least one compound of formula (I_{A}) corresponding to formula (I) in which R₁ represents a radical containing 18 carbon atoms, and from 90% to 10% by mass of at least one compound of formula (I_{B}) corresponding to formula (I), in which R₁ represents a radical containing 16 carbon atoms;
from 10% by mass to 95% by mass of a mixture of alkyl glyceryl ethers of formula (IIA) corresponding to formula (II) in which R₁ represents a radical containing 18 carbon atoms, and of alkyl glyceryl ethers of formula (IIB) corresponding to formula (II) for which R₁ represents a radical containing 16 carbon atoms.

5. Method of using a composition (C) as defined in claim 1, as an emulsifier.

6. A composition for topical use in the form of an oil-in-water emulsion, characterized in that it comprises, per 100% of its mass:
from 0.1% to 15% by mass of a composition (C) as defined in claim 1,
from 5% by mass to 50% by mass of a fatty phase formed from one or more oils and/or one or more waxes, and
from 94.9% by mass to 35% by mass of a cosmetically acceptable medium.

7. The composition as defined in claim 6,
characterized in that it also comprises, per 100% of its mass, from 0.1% by mass to 5% by mass of a thickening and/or gelling polymer.

8. The composition for topical use as defined in claim 6, characterized in that it also comprises, per 100% of its mass, from 0.5% by mass to 10% by mass of dihydroxyacetone.

9. A cosmetic treatment process for artificially tanning and/or browning the skin, characterized in that it consists in applying to the skin an effective amount of a composition as defined in claim 8.

10. Method for improving the stability of ire dihydroxyacetone contained in a cosmetic composition, in particular a cosmetic composition for artificially tanning and/or browning the skin, which comprises combining an effective amount of the composition defined in claim 1 to said cosmetic composition.
